(19) United States
(12) Patent Application Publication
(10) Pub. No.: US 2004/0133049 A1
(43) Pub. Date: Jul. 8, 2004

(54) Process for the continous fixed-bed hydrogenation of fatty acids and fatty acid esters

(76) Inventors: Christian Pelzer, Linnich (DE); Guenther Demmering, Solingen (DE); Udo Kreutzer, Monheim (DE); Hans-Peter Kubersky, Solingen (DE); Stephan Heck, Cologne (DE); Roland Hourticolon, Leichlingen (DE)

Correspondence Address: COGNIS CORPORATION, PATENT DEPARTMENT, 300 BROOKSIDE AVENUE, AMBLER, PA, 19002, US

(21) Appl. No.: 10/739,377

(22) Filed: Dec. 18, 2003

Related U.S. Application Data
(63) Continuation of application No. 09/167,467, filed on Oct. 7, 1998, now abandoned.

(51) Int. Cl. C07C 27/04

(52) U.S. Cl. 568/885

(57) ABSTRACT

Fatty acids, esters of fatty acids and naturally occurring triglycerides are continuously hydrogenated to fatty alcohols in a fixed-bed reactor in the presence of hydrogen in excess and hydrogenation catalysts under static pressures of 200 to 300 bar and at temperatures of 80 to 150° C. The liquid product is cooled and the excess hydrogen is returned to the reactor entrance by a gas circulation pump as a recycle gas after separation of the liquid product. The expense involved in cooling and reheating the recycle gas is eliminated without any reduction in the quality of the fatty alcohol produced providing the recycle gas is returned to the reactor entrance without reheating. The fatty alcohol produced contains a minimum amount of diol.

### BACKGROUND OF THE INVENTION

1. Field of the Invention

This invention relates to a process for the continuous hydrogenation of fatty acids and fatty acid esters using fixed-bed catalysts in which the hydrogen recycle gas is returned without reheating to the reactor entrance at temperatures of 80 to 150° C. and the hydrogenation reaction is carried out under static pressures of 200 to 300 bar to minimize the content of unwanted diols in the hydrogenation products.

2. Description of the Related Art

In the known process for the production of fatty alcohols, the starting material, for example fatty acids or fatty acid methyl esters, is introduced by pressure pumps into the installation where it is mixed with compressed hydrogen, heated with the compressed hydrogen to the reaction temperature and introduced into the reactor from above. Besides the ester group, carbon double bonds are also usually hydrogenated on the copper-containing catalyst, so that only saturated fatty alcohols are formed, even where unsaturated esters are used. Where the hydrogenation reaction is carried out using a copper-free catalyst on which the ester bond is selectively hydrogenated, unsaturated fatty alcohols are formed from unsaturated fatty acid esters or glycerides. EP-A 0 254 189 and EP-A 0 280 982, for example, are cited as representative of the extensive prior art literature available on the subject.

After passing through the reactors, the reaction mixture is cooled and is then separated in a separator into the liquid phase and the gas phase. The liquid phase is decompressed and passed to the methanol separation stage while the gas phase, which consists mainly of hydrogen, is circulated via a compressor. In the methanol separation stage which consists of an evaporator, the fatty alcohol is freed from the methanol and may then be put to its intended use without further purification.

A large excess of hydrogen, typically of the order of 100 to 200 moles of hydrogen per mole of ester, is characteristic of the known processes. The large amount of gas circulated requires considerable outlay on equipment, specially for cooling and reheating, which are normally carried out in several stages. After leaving the reactor, the gas/liquid mixture passes through a heat exchanger used to preheat the starting material. This is followed by cooling with water. After separation of the liquid phase, the recycle gas together with fresh hydrogen is introduced by a gas circulation pump into the feed pipe for the fatty compounds, mixed therewith and preheated in the heat exchanger mentioned. Finally, the mixture passes through a peak heater.

Unfortunately, the known processes are attended by serious disadvantages. On the one hand, cooling and reheating of the recycle gas involves considerable outlay on equipment, on the other hand the quality of the fatty alcohols in regard to the content of secondary products is not always satisfactory. Thus, fatty acid methyl esters which have been produced by transesterification of fats and oils always have a high percentage content of diols which are partial glycerides and which cannot be completely converted into the fatty alcohols under standard conditions. Instead, traces of these diols remain in the fatty alcohols and, for example in derivatives thereof such as ethoxylated fatty alcohols, can seriously impair performance properties, such as the cloud point for example, even in very small quantities.

Accordingly, the problem addressed by the present invention was to reduce the outlay required for cooling and reheating the recycle gas without any adverse effect on the quality of the fatty alcohol produced. More particularly, the invention set out to provide a process which would reliably minimize the content of unwanted diols in the hydrogenation products.

### SUMMARY OF THE INVENTION

Other than in the claims and in the operating examples, or where otherwise indicated, all numbers expressing quantities of ingredients or reaction conditions used herein are to be understood as modified in all instances by the term “about”.

A process for the continuous production of a saturated or unsaturated fatty alcohol by hydrogenation of a fatty acid, a fatty acid ester, or a triglyceride has been discovered. The process comprises contacting a fatty acid, a fatty acid ester, or a triglyceride with hydrogen at a pressure of from about 200 to about 300 bar and at a temperature of from about 80 to about 150° C. in the presence of a catalyst to form a liquid product phase comprised of at least a fatty alcohol having the same number of carbon atoms as the fatty acid, the fatty acid in the fatty acid ester, or the fatty acids in the triglyceride and unreacted hydrogen. The unreacted hydrogen is then separated from the liquid product phase and is recycled to the beginning of the process without reheating. Because the hydrogen is recycled without further heating from a temperature of from 80 to about 150° C., the process economics are considerably improved and the amount of unwanted diol in the product is significantly reduced.

### BRIEF DESCRIPTION OF THE DRAWING

FIG. 1 is a schematic representation of a commercial process for the hydrogenation of fatty acid methyl esters to fatty alcohols.

### DESCRIPTION OF THE PREFERRED EMBODIMENTS

The present invention relates to a process for the continuous fixed-bed hydrogenation of fatty acids and fatty acid esters to fatty alcohols, the liquid product being cooled and the excess hydrogen being returned as recycle gas to the reactor entrance by a gas circulation pump after separation of the liquid product, characterized in that the recycle gas is returned to the reactor entrance without reheating at temperatures in the range from 80 to 150° C. and the hydrogenation reaction is carried out under static pressures of 200 to 300 bar.

Although the recycle gas always remains at a temperature which experience has shown to correspond at least to the reactor entry temperature, it is nevertheless surprisingly possible to achieve high yields when the hydrogenation reaction is carried out under static pressures of at least 200 bar and preferably at least 270 bar. Not only the outlay on equipment, but also energy consumption are reduced. The invention includes the observation that the temperature window mentioned ensures that the content of unwanted diols is minimized.

Starting Materials

Suitable starting materials for the process according to the invention are fatty acids corresponding to formula (I):

R¹CO—OH (I)

in which R¹CO is an aliphatic, saturated or unsaturated, optionally hydroxy-substituted acyl radical containing 4 to 24 carbon atoms and preferably 12 to 18 carbon atoms. Typical examples are butyric acid, valeric acid, caproic acid, caprylic acid, 2-ethylhexanoic acid, capric acid, lauric acid, myristic acid, palmitic acid, palmitoleic acid, stearic acid, isostearic acid, oleic acid, elaidic acid, petroselic acid, linoleic acid, linolenic acid, elaeostearic acid, conjuene fatty acid, ricinoleic acid, arachic acid, gadoleic acid, behenic acid, erucic acid and brassylic acid and the technical mixtures thereof obtained, for example, in the pressure hydrolysis of natural fats and oils or as monomer fraction in the dimerization of unsaturated fatty acids.

It is known that the use of fatty acids in the hydrogenation reaction involves increased outlay on equipment because the reactors have to be protected against corrosion and the catalysts against salt formation. Accordingly, the fatty acids are preferably replaced by their esters with lower alcohols containing 1 to 4 carbon atoms, preferably methyl esters of the fatty acids mentioned above. However, the methyl esters may also be replaced by the corresponding triglycerides, i.e. the refined, hydrogenated or non-hydrogenated fats and oils, for example palm oil, palm kernel oil, coconut oil, cottonseed oil, peanut oil, rapeseed oil (from old and new plants), sunflower oil (from old and new plants), olive oil, olive kernel oil, coriander oil, castor oil, meadowfoam oil, chaulmoogra oil, tea seed oil, linseed oil, beef tallow, lard, fish oil and the like.

The present invention also includes the observation that there is a critical limit to the partial glyceride concentration in the starting material for the formation of diols in the hydrogenation reaction. Accordingly, starting materials with a partial glyceride concentration of less than 0.5% by weight and preferably less than 0.44% by weight are preferred.

Catalysts

Suitable catalysts are, generally, Adkins catalysts which are mixed oxidic copper/chromium, copper/zinc or copper/aluminium oxides. Examples of suitable catalysts can be found in DE-A 37 06 658, DE-A 39 13 387, DE-A 40 05 629, DE-A 40 00 692, DE-A 41 29 622, DE-A 42 42 466, DE-A 43 20 460 and DE-A 43 21 837 (Henkel) and in the review article by M. Schneider et al. in Fat Sci. Technol. 89 508 (1987).

Reactors

Since the hydrogenation reaction is exothermic, the heat of reaction is dissipated either by cooling the reactor or—outside the reactor—by cooling the recycle gas. The heat dissipated may advantageously be used to preheat or peak-heat the starting product. Thus, in one advantageous embodiment, the hydrogenation reaction is carried out in a cooled tube bundle reactor or tube reactor. The recycle gas is preferably returned to the reactor without cooling. The heat of reaction is dissipated solely by cooling the reactor.

However, the process according to the invention may also be carried out in a shaft reactor. In this case, the hydrogenation reaction is carried out in an uncooled shaft reactor and, after leaving the reactor and before or after separation of the liquid product, the recycle gas is cooled to dissipate the heat of reaction. In contrast to the prior art, however, the issuing recycle gas is not cooled more than necessary for dissipating the heat of reaction. The process according to the invention may be carried out both in countercurrent and in co-current.

Carrying out the Process

Since, according to the invention, the recycle gas is cooled only slightly, if at all, the heat to be dissipated can no longer be used to preheat the liquid starting product as in the prior art. In order nevertheless to minimize energy consumption, the liquid product is cooled—more particularly after separation of the gas phase—in a heat exchanger which preheats the liquid starting material.

In the process according to the invention, the hot hydrogen gas under pressure is circulated by the gas circulation pump. The high temperatures involved mean that the pump has to be correspondingly designed. This may be done simply and economically by recirculating the recycle gas using a piston pump of which the cylinder and piston are connected to the valves by pendulum lines of which the volume is at least three times the swept volume of the pump. In these pendulum lines, there is no continuous flow of gas, merely a back and forth swinging movement. Accordingly, the hot working valves of the pump can be arranged at a sufficient distance from the cylinder and piston with their sensitive packings and drive elements. Although it transports a hot gas, the pump—except for the valves—may be kept at a relatively low temperature. A corresponding circulation process for hot gases, more especially under high pressures, is known from DE-AS 10 44 343 and from DE-PS 10 48 665 and also from DE-PS 10 77 367. Reference is specifically made at this juncture to the disclosures of these documents.

In contrast to the known and conventional hydrogenation process, the recycle gas contains a relatively high percentage of water if a fatty acid is used as the starting material or methanol if a methyl ester of a fatty acid is used as the starting material. According to the invention, the resulting shift in the equilibrium reaction to the side of the starting materials can be compensated by maintaining minimum pressures so that unexpectedly high yields are still obtained.

It has also surprisingly been found that the reaction temperature in the process according to the invention can be considerably lower than in the known processes mentioned above without having to accept a reduction in the conversion as expressed by the residual saponification value. Thus, the hydrogenation reaction for the production of saturated fatty alcohols is preferably carried out at reaction temperatures of 190 to 220° C. The relatively low tendency towards the formation of secondary products and the lower temperatures to which the catalyst is exposed are of advantage in this regard.

The following examples are meant to illustrate but not to limit the invention.

### Examples

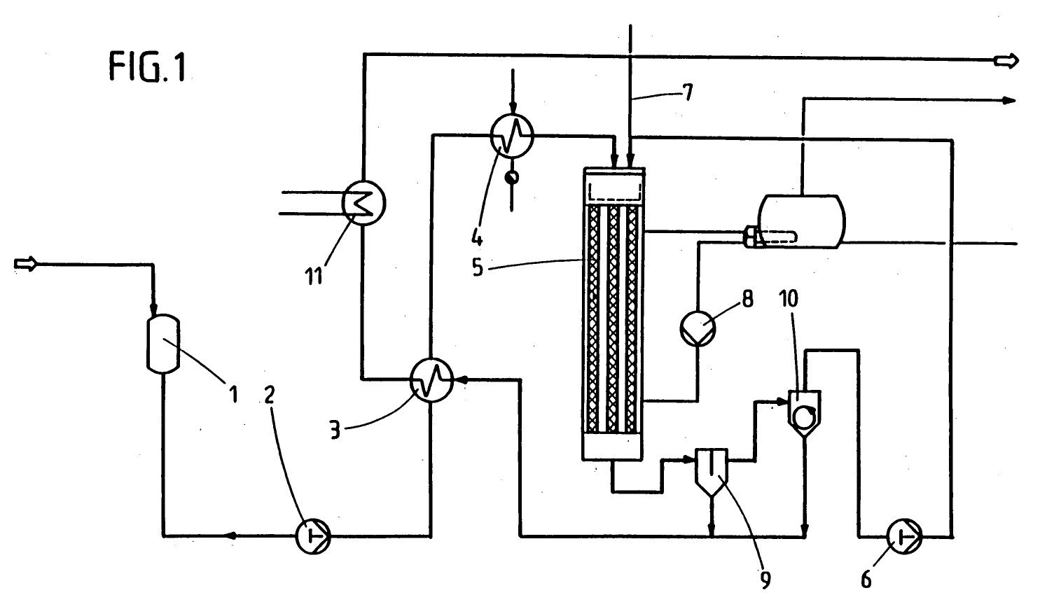

Test results obtained with an industrial hydrogenation plant of the type shown in FIG. 1 are explained in the following. The reference numerals used have the following meanings: 1 holding vessel 2 pressure pump 3 heatexchanger 4 peak heater 5 reactor 6 gas circulation pump 7 feed pipe for fresh hydrogen 8 pump 9 separator 10 separator 11 cooler

Description of a Commercial Process

FIG. 1 schematically illustrates a commercial process for the hydrogenation of fatty acid methyl esters to fatty alcohols. The starting material is introduced into the plant from a holding tank 1 by a pressure pump 2, preheated in a heat exchanger 3, brought to the reaction temperature by a steam-operated peak heater 4 and introduced into a tube bundle reactor 5 from above. The recycle gas together with fresh hydrogen (feed pipe 7) is also delivered to the head of the reactor 5 by a gas circulation pump 6. A pump 8 passes a heat transfer oil through the reactor 5 for cooling. The effluent from the reactor 5, a gas/liquid mixture, is separated in the separators 9 and 10 and, after cooling in the heat exchanger 3 and a following cooler 11 operated with cooling water, is delivered to the methanol separation stage. The gas phase is returned as recycle gas to the reactor 5 by the gas circulation pump 6.

### Examples 1 to 6, Comparison Example C1

Table 1 shows the dependence of the diol content on the recycle gas temperature and the reaction temperature where the hydrogenation plant is operated under isothermal conditions. Saturated fatty alcohol was produced from palm kernel oil fatty acid methyl ester with a partial glyceride content of 2.22% by weight in the presence of a copper/zinc catalyst. In Comparison Example C1, the recycle gas temperature is above the critical range. It can be seen that the diol content is not significantly minimized here. The Examples according to the invention were carried out at recycle gas temperatures of 100 to 135° C. The diol content was more than halved in relation to the Comparison Example.

TABLE 1
Influence of the recycle gas temperature on the diol content

| | LHSV¹ | H₂ | | | | | | |
|---|---|---|---|---|---|---|---|---|
| Ex. | (hr⁻¹) | (Dm³/h) | T² | p³ | T⁴ | SV | DIOL⁵ | US⁶ |
| C1 | 1.50 | 170 | 190 | 270 | 170 | 0.95 | 0.45 | 0.15 |
| 1 | 1.50 | 170 | 210 | 270 | 100 | 0.85 | 0.15 | 0.20 |
| 2 | 1.50 | 170 | 215 | 270 | 100 | 0.90 | 0.12 | 0.22 |
| 3 | 1.50 | 170 | 220 | 270 | 100 | 0.95 | 0.06 | 0.40 |
| 4 | 1.50 | 170 | 210 | 270 | 135 | 0.85 | 0.23 | 0.21 |
| 5 | 1.50 | 170 | 215 | 270 | 135 | 0.92 | 0.17 | 0.22 |
| 6 | 1.50 | 170 | 220 | 270 | 135 | 1.00 | 0.09 | 0.42 |

Legend:
¹Liquid hourly space velocity
²Reaction temperature
³Reaction pressure
⁴Recycle gas temperature
⁵Diol in % by weight
⁶Unsaponifiables in % by weight

What is claimed is:

1. A process for the continuous production of a saturated or unsaturated fatty alcohol comprising the steps of: (1) contacting a fatty acid or fatty acid ester with hydrogen at a pressure of from about 200 to about 300 bar and at a temperature of from about 80 to about 150° C. in the presence of a catalyst to form a liquid product phase and unreacted hydrogen; (2) separating said liquid product phase from said unreacted hydrogen; (3) without reheating, recycling said unreacted hydrogen to step (1).

2. The process of claim 1 wherein said fatty acid ester is an ester of a fatty acid having from about 6 to about 22 carbon atoms and a lower alcohol having 1 to 4 carbon atoms.

3. The process of claim 1 wherein the glyceride content of said fatty acid or fatty acid ester is less than about 0.5% by weight.

4. The process of claim 1 wherein said catalyst is an oxidic copper/chromium, copper/zinc or copper/aluminium catalyst.

5. The process of claim 1 wherein said process is carried out in a cooled tube bundle reactor or tube reactor.

6. The process of claim 1 wherein said recycle gas is returned to the reactor without cooling.

7. The process of claim 1 wherein said process is carried out in an uncooled shaft reactor and, after leaving the reactor and before or after separation of the liquid product, the recycle gas is cooled to dissipate the heat of reaction.

8. The process of claim 1 wherein said liquid product phase is cooled in a heat exchanger which preheats the liquid starting material.

9. The process of claim 1 wherein said recycle gas is circulated by a piston pump of which the cylinder and piston are connected to the valves by pendulum lines of which the volume is at least three times the swept volume of the pump.

10. The process of claim 1 wherein step (1) is carried out at a temperature of from about 190 to about 220° C.
